(19) United States
(12) Patent Application Publication
(10) Pub. No.: US 2015/0330978 A1
(43) Pub. Date: Nov. 19, 2015

(54) TARGETED MORPHOLINO LIGANDS AND USES THEREOF

(71) Applicant: Vivonics, Inc., Waltham, MA (US)

(72) Inventor: Vladimir Leo Gilman, Westford, MA (US)

(73) Assignee: VIVONICS, INC., Waltham, MA (US)

(21) Appl. No.: 14/278,866

(22) Filed: May 15, 2014

(51) Int. Cl. G01N 33/569; C07F 9/6558

(52) U.S. Cl. CPC G01N 33/569; C07F 9/65583

(57) ABSTRACT

Morpholino ligands that have high specificity for targets, such as proteins, and that bind their targets through non Watson-Crick base pairing, are described. The use of such morpholino ligands for targeted delivery of loads, such as drugs or labels, are disclosed.

### FIELD OF THE INVENTION

The invention generally relates to morpholino ligands that specifically bind to target molecules through non Watson-Crick base pairing, such as proteins.

### BACKGROUND

Aptamers are nucleic acid macromolecules that specifically bind to target molecules. Like all nucleic acids, a particular nucleic acid ligand, i.e., an aptamer, may be described by a linear sequence of nucleotides (A, U, T, C and G), typically 15-40 nucleotides long. In solution, the chain of nucleotides forms into a complex three-dimensional shape due to intramolecular interactions. Because of the three-dimensional shape of the nucleic acid ligand, it is able to bind to targets, e.g., proteins and polysaccharides, that have complimentary three-dimensional structures, rather than binding via Watson and Crick pairing. An aptamer's specificity also results in a consummate lack of interaction with non-target molecules. Furthermore, because aptamers primarily comprise naturally-metabolized materials, i.e., nucleic acids, excess aptamers that do not specifically-bind to targets are rapidly cleared from the body.

It is theorized that the aptamers act much like paratopes of an antibody. The three-dimensional structure of the aptamer is uniquely suited (“key”) to bind to a matching epitope (“lock”) on a target. That specificity results in high selectivity for the target molecules, typically proteins or polysaccharides. Aptamers have been used to target specific proteins and deliver blocking compounds, such as polyethylene glycol polymers, to targets. See, for example, pegaptanib (brand name Macugen), an anti-angiogenic for treatment of age-related macular degeneration, configured to bind to a particular isoform of the VEGF protein.

In practice, aptamers have drawbacks when used as ligands. Particularly, the negatively-charged phosphate backbone of the nucleic acid chain facilitates electrostatic attraction and cross-reactivity where it is not intended. Thus, in some cases, nucleic acid aptamers will non-specifically bind various positively charged species found in a biological environment. The amount of non-specific binding is most problematic when the positively charged species are present at levels exceeding those of an anticipated target, or when the aptamer has to interact with a good number of positively charged species on the way to the target.

### SUMMARY

The invention recognizes that aptamer non-specific binding is caused by significant negative electrostatic charge of an aptamer under physiological conditions. The negative charge comes from phosphate residues connecting individual nucleosides of an aptamer sequence. Thus, aptamers non-specifically bind various positively charged species found in biological samples. That is especially true if the positively charged species are present at levels exceeding those of an anticipated target, which can easily be affected by slight changes in pH and salinity, both of which are common in complex biological fluids.

To address that problem, the invention provides isolated and synthesized morpholino ligands that are electrostatically neutral under physiological conditions. The morpholino ligands of the invention can be used to specifically bind to targets, such as proteins, through non Watson-Crick base pairing, because the morpholino ligands have a macromolecular structure that complements a binding site on the target. Similar to an aptamer, the morpholino ligands include a plurality of individual units, generally resembling nucleic acid oligomers and the morpholino ligands of the invention bind the target based on three-dimensional structure of the target, rather than Watson-Crick base paring. However, unlike aptamers, the sugar phosphate backbone that would normally be present in the nucleic acid oligomer is replaced with morpholine (diethylenimide oxide) units coupled with phosporoamidate linkers. That structure results in a ligand that is electrostatically neutral under physiological conditions, thereby overcoming the problems associated with in vivo use of aptamers, such as non-specific binding interactions with positively charged non-target molecules due to negative charge on the aptamer from the negatively charged phosphate groups. Because the morpholino structure is closely related to nucleic acid aptamers, it is straightforward to identify morpholino molecules that will specifically-bind an identified target.

In many instances, the morpholino ligands will be linked to a load, e.g., a chemical moiety, e.g., a drug or a label, to allow that load to be delivered to a specific target. The load may be linked to the morpholino ligand with any linker known in the art, for example an enzyme cleavable linker. In some embodiments, morpholino ligands of the invention may be linked to a drug. In other embodiments, the load may be a detectable label, such as an optically detectable label, such as a fluorescent label, or a radiopaque label. The morpholino ligands, thus, lend themselves to drug delivery systems and label delivery systems.

### BRIEF DESCRIPTION OF THE DRAWINGS

FIG. 1 depicts a generalized morpholino molecule that can be used as a specific ligand according to the invention.

FIG. 2 is a schematic representation of a general procedure for identification of a specifically-binding morpholino ligand. In step (a) a random morpholino oligo library is exposed to a desired target, and an affinity candidate is retained by an epitope of this target. Next, the morpholino candidate is detached from the target in step (b). The morpholino candidate is then mixed with a library of random RNA oligonucleotides in step (c). The mixture is then separated by size in an agarose gel to isolate morpholino-RNA complexes formed via the Watson and Crick pairing. These complexes are extracted from the appropriate gel segments, and the RNA is translated into a cDNA construct. See step (d). The cDNA sequence is determined by traditional DNA sequencing methods. The morpholino base sequence is then deduced by the complementarity rules according to the sequenced cDNA construct. See step (e). The identified morpholino is then characterized for its affinity and specificity to the target.

FIG. 3 illustrates a delivery system including a targeted morpholino ligand.

### DETAILED DESCRIPTION

The invention describes the use of morpholino ligands to target specific structures, such as proteins and polysaccharides, and delivery systems, such as drug or label delivery systems that incorporate the morpholino ligands. The target may be an in vivo target or an in vitro target. The macromolecular structure of the morpholino ligands allows the ligands to specifically-bind to epitopes of the target that have complimentary macromolecular structures. Specifically, the morpholinos described in the invention are useful for specifically binding with targets because of steric configurations and not Watson-Crick pairing, i.e., to a nucleic acid. However, because the morpholino molecules are generally electrostatically neutral at physiological conditions, the morpholino molecules undergo less non-specific binding than similar aptamer ligands.

Morpholinos were initially designed as synthetic molecules that would mimic Watson-Crick nucleic acid pairing, but would not interact with enzymes involved in the transcription process. Accordingly, they are widely used to “knock out” genes in an organism to determine function of those genes, e.g., what proteins a particular gene transcribes. Morpholinos, in many ways, resemble nucleic acids, such as deoxyribonucleic acid (DNA) and ribonucleic acid (RNA), however the sugar phosphate backbone found in DNA and RNA has been replaced with morpholine rings that are linked with a backbone, typically phosphate or a phosphate derivative, such as phosphoroamidate. An exemplary morpholino unit including a phophoroamidate backbone is shown below.

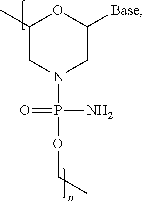

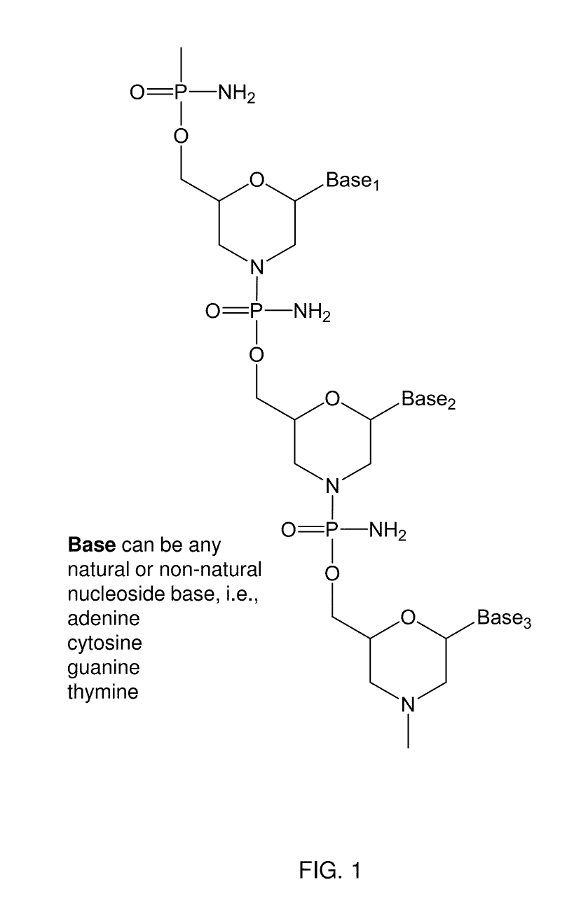

The backbone of the morpholinos of the invention may be phosphoroamidate, phosphate or another phosphate derivative, such as a phosphoester. In preferred embodiments, the backbone is phosphoroamidate because it results in a backbone with a more homogenous electron density and thus less likely to undergo non-specific binding. An exemplary morpholino ligand, including three bases is shown in FIG. 1.

Morpholinos can be or any useful length. In other words, n is only limited by the usefulness of an oligomer of that length. In most cases, n is less than 200, e.g., less than 150, e.g., less than 120, e.g., less than 100, e.g., less than 80, e.g., less than 50. In most cases, n is greater than 2, e.g., greater than 5, e.g., greater than 10, e.g., greater than 15, e.g., greater than 25, e.g., greater than 50. For example, n can be 2, 3, 4, 5, 10, 15, 20, 25, 30, 40, 50, 60, 70, 80, 90, 100, 200, 300, 500, 1000 etc., and any number between those exemplified numbers.

Because morpholinos are typically used for knock-out and antisense experiments, i.e., to bind to DNA and RNA, the bases connected to the morpholine ring typically include naturally occurring nucleic acid bases, such as adenine, cytosine, guanine, thymine, and uracil. However other naturally occurring or non-naturally occurring bases may be incorporated into morpholinos, such as isoguanine, inosine, thiouridine, pseudouridine, dihydrouridine, queuosine, and wyosine. For anti-sense experiments, commercial morpholinos are typically about 25 bases in length. In some embodiments, additional chemical functionalities are included in the morpholino ligands, and result increased affinity (as compared to the non-modified morpholino ligands) due to additional capabilities of binding to the anticipated targets via these additional functionalities.

Morpholino ligands are commercially available from suppliers, such as Gene Tools, LLC (Philomath, Oreg.). They are typically synthesized by using phosphoramidate blocking and deblocking steps on a template attached to a solid support. See, e.g., N. D. Sinha et al. “Polymer support oligonucleotide synthesis XVIII: use of beta-cyanoethyl-N,N-dialkylamino-/N-morpholino phosphoramidate of deoxynucleosides for the synthesis of DNA fragments simplifying deprotection and isolation of the final product,” Nucleic Acids Research. Jun. 11, 1984; 12(11): 4539-4557, incorporated by reference in its entirety.

Morpholinos, such as morpholinos described above, can be used as target-specific ligands for delivering loads, e.g., chemical moieties, e.g., drugs or labels, to a target, such as a protein. The targeted molecules can be any molecule identified as having a high specific binding with a morpholino. The target may be in vivo or in vitro. Morpholino ligands and targets can be matched using known screening techniques, such as the screening assay described below. The specificity of the binding can be described with a binding constant that describes the preference for a ligand and a target to be bound together rather than separate, e.g., under physiological conditions. The ligand target binding can be generally described as

L+T [symbol] L·T

and characterized with a binding constant

$${K}_{b} = \frac{[ L \cdot T ]}{[ L ] [ T ]} .$$

(The binding constant is properly reported in reciprocal concentration units, such as L/mol, however the units are typically not as important as the scale of the K_{b}. Accordingly, the units have been dropped in this disclosure.) Morpholino ligands suitable for use with the invention typically have binding constants of at least 1×10⁶, such as at least 1×10⁷, such as at least 1×10⁸, such as at least 1×10⁹, such as at least 1×10¹⁰.

Because the morpholinos are electrostatically neutral, non-specific binding due to electrostatic attraction is greatly reduced. That is, neutral morpholinos have no electric charge, i.e., no moiety in the morpholino possessing a charge under physiological conditions. (Normal physiological conditions in the human body refers to normal conditions within mammalian tissue or body fluid under which biological reactions occur in the absence of environmental stressors. Normal physiological conditions are generally a pH of about 7 to about 8, preferably between 7.3 and 7.6, and a temperature of about 35° C. to about 38° C., preferably 37° C. Furthermore, the normal concentration of sodium in the blood plasma is 136-145 mM.)

Morpholinos suitable for use with the invention can be determined by screening methods that are used to identify aptamers. For example, SELEX as described in Gold et al. (U.S. Pat. No. 5,270,163) can be used to identify morpholino ligands that specifically bind a target, such as an in vivo target, via non Watson-Crick based pair, such as based on the conformational shape of the morpholino ligand. Other methods are shown in Gilman et al. (U.S. patent application number 20110104667, incorporated herein by reference in its entirety).

After separation, the nucleotide sequence of the morpholino ligands of the invention may be obtained via sequencing the cDNA reverse transcripts (obtained i.e. using Life Technologies SuperScript® III Reverse Transcriptase: Evolution of the SuperScript® Reverse Transcriptases. http://www.lifetechnologies.com/us/en/home/life-science/per/reverse-transcription/reverse-transcriptase-enzymes/superscript-iii-reverse-transcriptase.html. Reverse transcriptases useful in the invention include, but are not limited to, reverse transcriptases from HIV, HTLV-1, HTLV-II, FeLV, FIV, SIV, AMV, MMTV, MoMuLV and other retroviruses (see Levin, Cell 88:5-8 (1997); Verma, Biochim Biophys Acta. 473:1-38 (1977); Wu et al., CRC Crit Rev Biochem. 3:289-347(1975)). cDNA sequencing may be by any method known in the art. See for example Sanger et al. (Proc Natl Acad Sci USA, 74(12): 5463 67, 1977), Maxam et al. (Proc. Natl. Acad. Sci., 74: 560-564, 1977), and Drmanac, et al. (Nature Biotech., 16:54-58, 1998), which references describe exemplary conventional ensemble sequencing techniques. Also see Lapidus et al. (U.S. Pat. No. 7,169,560), Quake et al. (U.S. Pat. No. 6,818,395), Harris (U.S. Pat. No. 7,282,337), Quake et al. (U.S. patent application number 2002/0164629), and Braslaysky, et al., (PNAS (USA), 100: 3960-3964, 2003), which references describe exemplary single molecule sequencing by synthesis techniques. The contents of each of the references is incorporated by reference herein in its entirety.

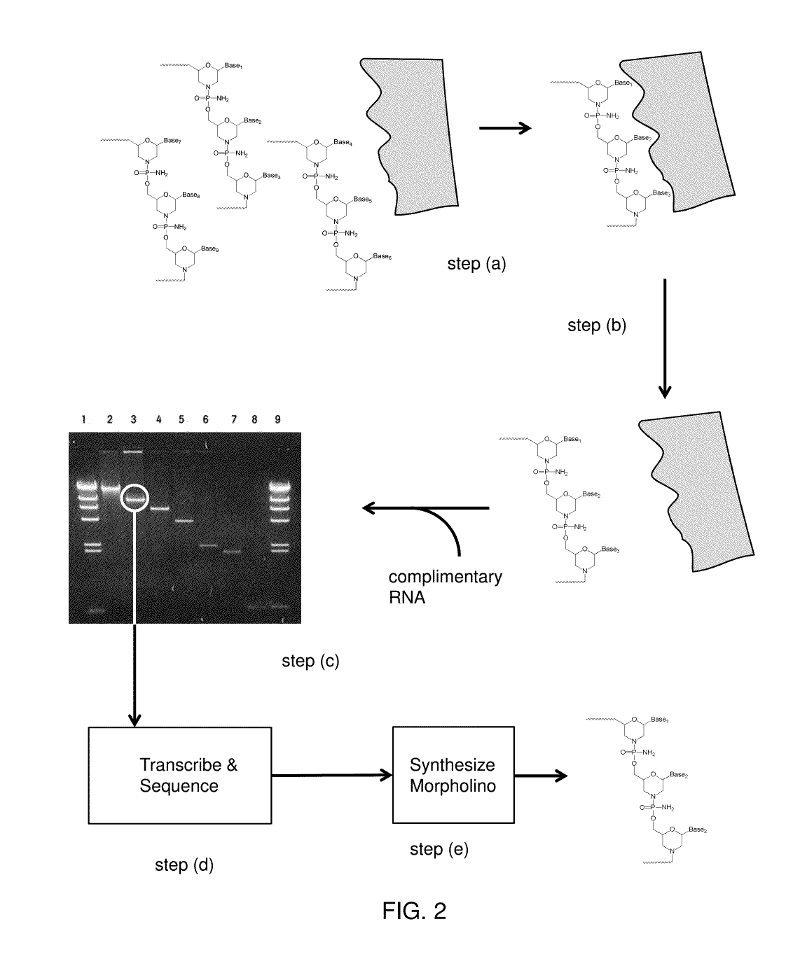

A method for identifying a specifically-binding morpholino is shown in FIG. 2. In step (a), a random morpholino oligo library is exposed to the desired target and an affinity candidate is retained by an epitope of this target. The morpholino-oligo library may be a commercially available library, or the library may be custom produced, for example from a supplier such as Gene Tools, LLC (Philomath, Oreg.). Both the target and the library may be in solution, or the target may be bound to a solid support, or the morpholino oligos may be bound to a solid support, such as an array. Members of the library or the target may be labeled with one or more fluorescent markers.

After the target is exposed to the library, the non-binding library members are removed and the affinity candidate is detached from the target, as shown in step (b) of FIG. 2. Once isolated, the morpholino affinity candidate structure is determined by complexing the morpholino with complimentary RNA oligonucleotides provided in a library mixture. The double-stranded morpholino RNA complexes are isolated, for example using an electrophoretic gel, as shown in step (c). Alternatively, the double-stranded morpholino RNA complexes can be isolated with other methods of separation or chromatography. Once separated, the complimentary RNA is transcribed with reverse transcription to create corresponding cDNA transcripts, as shown in step (d). The cDNA is then sequenced with any sequencing technique, such as Sanger sequencing or next generation sequencing, e.g., Illumina sequencing. Other suitable sequencing techniques, known to those of skill in the art, can be used to identify a preferred nucleic acid sequence. Finally, once the preferred aptamer sequence is identified, the corresponding morpholino oligo is synthesized (step (e)) and characterized for its affinity and specificity to the target. In some instances the morpholinos may be “off-the-shelf” commercially-available for use in gene knock-out experiments. In other instances, the desired morpholinos will have to be synthesized. Such synthesis is commercially available from Gene Tools, LLC.

The techniques for preparing cDNA from RNA are generally known in the art. Typically, the technique comprises synthesizing a cDNA strand from an RNA template using a reverse transcriptase to yield a plurality of cDNA strands of varying read length. The various strands are then sequenced using any known sequencing technique, e.g., Sanger sequencing or next-generation sequencing. By controlling the extent of the reverse transcriptase reaction by methods known in the art, the resulting reads will have a variety of start positions. This allows for increase accuracy, especially in long mRNA templates. Controlling the reverse transcriptase reaction also allows more informative counting (i.e., increased variability in start sites leads to more informative counting). The variability in read length introduced by this method also facilitates focus on the most accurate sequencing reads.

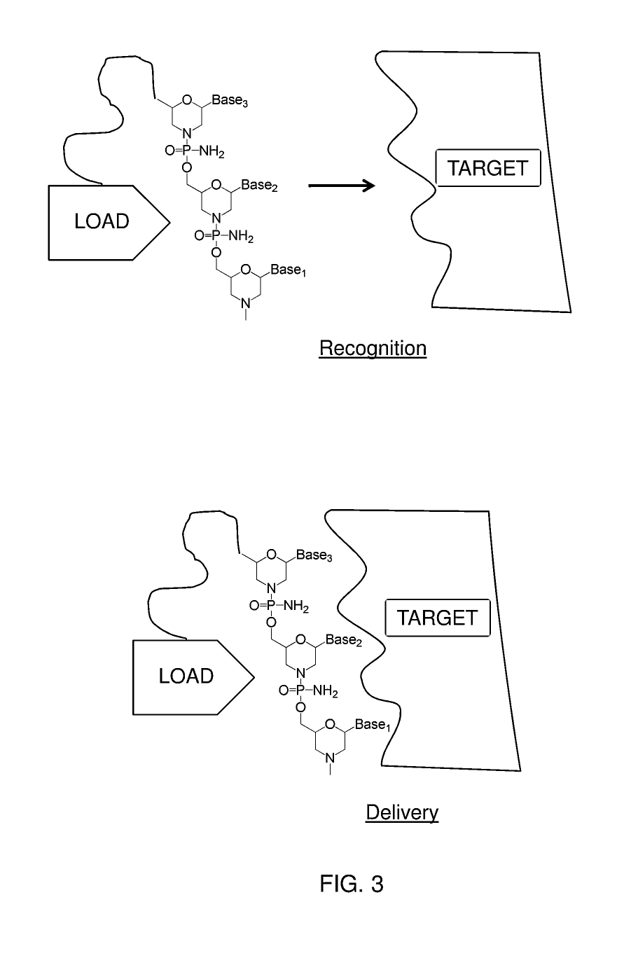

Once a specifically-binding morpholino ligand has been identified, that morpholino ligand can be used for a variety of tasks. In a preferred embodiment, the morpholino ligand will be linked to a load to be delivered to a protein, as shown in FIG. 3. For example, a drug may be coupled to the morpholino ligand for delivery to a biological structure having that protein. For example, a surface protein expressed by a cancer cell may be the binding target for the morpholino ligand, and the drug may be an anti-angiogenic agent or an anti-neoplastic agent. In other embodiments, the morpholino ligand can be coupled to a label, such as a fluorescent label, a radiopaque label, a radioactive label, a chemoluminescent label, a luminescent label, a phosphorescent label, a fluorescence polarization label, or a charge label. In other embodiments, the morpholino ligand can be coupled to a molecule that will interact with directed energy from a directed energy machine, e.g., a proton beam machine, thus delivering therapy to the target.

The methods and delivery systems of the invention can be used generally with drugs and therapeutics that are to be delivered to a target. Such drugs include anti-infectives, such as antibiotics and antiviral agents, analgesics and analgesic combinations, anti-inflammatory agents, local and general anesthetics, sedatives, antipsychotic agents, neuroleptic agents, antidepressants, anxiolytics, neuron blocking agents, anticholinergic and cholinomimetic agents, antimuscarinic and muscarinic agents, antiadrenergics, antiarrhythmics, antihypertensive agents, hormones, and nutrients, antiarthritics, antiasthmatic agents, anticonvulsants, antihistamines, antinauseants, antineoplastics, antipruritics, antipyretics, antispasmodics, cardiovascular preparations (including calcium channel blockers, beta-blockers, beta-agonists and antiarrythmics), antihypertensives, diuretics, vasodilators, central nervous system stimulants, decongestants, diagnostics, hormones, bone growth stimulants and bone resorption inhibitors, immunosuppressives, muscle relaxants, psychostimulants, sedatives, tranquilizers, and small molecules for specialized treatments. Therapeutics may also biologics, including cells (e.g., stem cells), proteins (e.g., enzymes), and vaccines.

In certain embodiments, the detectable label is a fluorescent label. Suitable fluorescent labels include, but are not limited to, 4-acetamido-4′-isothiocyanatostilbene-2,2′disulfonic acid; acridine and derivatives: acridine, acridine isothiocyanate; 5-(2′-aminoethyl)aminonaphthalene-1-sulfonic acid (EDANS); 4-amino-N[3-vinylsulfonyl)phenyl]naphthalimide-3,5 disulfonate; N-(4-anilino-1-naphthyl)maleimide; anthranilamide; BODIPY; Brilliant Yellow; coumarin and derivatives; coumarin, 7-amino-4-methylcoumarin (AMC, Coumarin 120), 7-amino-4-trifluoromethylcouluarin (Coumaran 151); cyanine dyes; cyanosine; 4′,6-diaminidino-2-phenylindole (DAPI); 5′5″-dibromopyrogallol-sulfonaphthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansylchloride); 4-dimethylaminophenylazophenyl-4′-isothiocyanate (DABITC); eosin and derivatives; eosin, eosin isothiocyanate, erythrosin and derivatives; erythrosin B, erythrosin, isothiocyanate; ethidium; fluorescein and derivatives; 5-carboxyfluorescein (FAM), 5-(4,6-dichlorotriazin-2-yl)aminofluorescein (DTAF), 2′,7′-dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; o-phthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of sulforhodamine 101 (Texas Red); N,N,N′,N′tetramethyl-6-carboxyrhodamine (TAMRA); tetramethyl rhodamine; tetramethyl rhodamine isothiocyanate (TRITC); riboflavin; rosolic acid; terbium chelate derivatives; Cy3; Cy5; Cy5.5; Cy7; IRD 700; IRD 800; La Jolla Blue; phthalo cyanine; and naphthalo cyanine.

The fluorescently labeled bases may be obtained commercially (e.g., from NEN DuPont, Amersham, and BDL). Alternatively, fluorescently labeled nucleotides may also be produced by various techniques, such as those described in Kambara et al. (Bio/Technol., 6:816-21, 1988); Smith et al. (Nucl. Acid Res., 13:2399-2412, 1985); and Smith et al. (Nature, 321: 674-679, 1986). Extensive guidance exists in the literature for derivatizing fluorophore and quencher molecules for covalent attachment via common reactive groups that may be added to a nucleotide base. Many linking moieties and methods for attaching fluorophore moieties to nucleotides also exist, as described in Oligonucleotides and Analogues, supra; Guisti et al., supra; Agrawal et al, supra; and Sproat et al., supra.

In many embodiments, the morpholino ligand will be linked to the load, e.g., a drug or label with a linker. The linker may be cleavable, e.g., by a naturally occurring enzyme. The linker may comprise an alkylene chain, a polyethylene glycol (PEG) chain, polysuccinic anhydride, poly-L-glutamic acid, poly(ethyleneimine), an oligosaccharide, or an amino acid chain. Various oligomeric linker groups that are biologically compatible and/or bioerodible are known in the art, and the selection of the linkage may influence the ultimate properties of the combined load and morpholino ligand, such as whether it is durable when implanted, whether it gradually deforms or shrinks after implantation, or whether it gradually degrades and is absorbed by the body. The linker group may be attached to the moieties by any suitable bond or functional group, including carbon-carbon bonds, esters, ethers, amides, amines, carbonates, carbamates, sulfonamides, etc.

Certain chemical modifications of the morpholino ligands of the invention may be made to increase the in vivo stability of the morpholino ligand or to enhance or to mediate the delivery of the morpholino ligand. See, e.g., Pieken et al. (U.S. Pat. No. 5,660,985), the contents of which are incorporated by reference herein in their entirety. Modifications of the morpholino ligands contemplated in this invention include, but are not limited to, those that provide other chemical groups that incorporate additional charge, polarizability, hydrophobicity, hydrogen bonding, electrostatic interaction, and functionality to the morpholino ligand bases or to the morpholino ligand as a whole. Such modifications include, but are not limited to morpholine ring modifications, substitution of sulfur for oxygen in the morpholine ring, backbone modifications, such as phosphorothioate or alkyl phosphate modifications, methylations, unusual base-pairing combinations such as the isobases isocytidine and isoguanidine and the like. Modifications can also include 3′ and 5′ modifications such as capping.

Thus, as described above, the morpholino ligands of the invention specifically bind target molecules by matching a macromolecular configuration of the morpholino ligand with a macromolecular configuration of the target. The morpholino ligands can, thus, be used to deliver loads to the targeted molecules. The described delivery platforms of the invention are importantly different from prior art delivery platforms that deliver loads to specific nucleic acid sequences with targeted Watson and Crick pairing. Because the morpholino ligands do not employ Watson and Crick pairing, they can be used to target a wider variety of targets, such as proteins and polysaccharides.

In certain embodiments, the morpholino ligand binds to CD271. Nucleic acid ligands that bind to CD271 are described for example in Gilman (PCT number PCT/US 14/19284), the content of which is incorporated by reference herein in its entirety. Based on the known sequence of those nucleic acid ligands, morpholino ligands have that same sequence can be synthesized. The advantage of the morpholino ligands is that they will be neutral under physiological conditions as compared to the aptamers. The morpholino ligands can be coupled to a drug for targeted to delivery to cells that express CD271, such as cancer cells. In other embodiments, the morpholino ligands may be part of an implantable medical product, that includes a scaffold composed of a biocompatible material, and a plurality of morpholino ligands that binds to CD271. Once implanted, the morpholino ligands will attract adult stem cells that express CD271. The increased rate of adult stem cell retention results in increased density of somatic tissue cells generated on the surface of the implant, providing an increased rate of tissue regeneration.

In certain embodiments, the morpholino ligand binds to an infectious prion. Nucleic acid ligands that bind to infectious prion are described for example in Gilman (U.S. patent application publication number 2011/0104668), the content of which is incorporated by reference herein in its entirety. Based on the known sequence of those nucleic acid ligands, morpholino ligands have that same sequence can be synthesized. The advantage of the morpholino ligands is that they will be neutral under physiological conditions as compared to the aptamers.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A method of specifically binding a moiety to a target, comprising:
providing a morpholino ligand that is coupled to a moiety, the morpholino ligand comprising Formula I that specifically binds a target, wherein Formula I is: 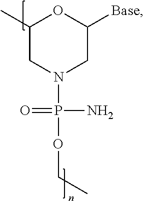
Base is any natural or non-natural base, and n is an integer between 2 and 200; and
contacting the target with the identified morpholino ligand coupled to the moiety, wherein the morpholino ligand specifically binds the target via non Watson-Crick binding.

2. The method of claim 1, wherein the target is a protein.

3. The method of claim 2, wherein the protein is located on the surface of a cell.

4. The method of claim 1, wherein the moiety is a therapeutic, a label, or a marker.

5. The method of claim 4, wherein the label comprises a fluorescent molecule.

6. The method of claim 1, wherein the identified morpholino ligand is coupled to the moiety with a cleavable linker.

7. The method of claim 6, wherein the cleavable linker is enzymatically cleavable.

8. The method of claim 1, wherein the identified morpholino ligand binds the target with a binding constant of greater than 1×10⁶.

9. The method of claim 1, wherein Base is selected from adenine, cytosine, guanine, and thymine.

10. The method of claim 1, wherein n is an integer selected from the group consisting of: greater than 10, greater than 25, and greater than 50.

11. The method of claim 1, wherein prior to the providing step, the method further comprises identifying the morpholino ligand by screening a library of morpholino oliogomers against the target.

12. The method of claim 11, further comprising reverse transcribing an RNAoliogmer that is complimentary the identified morpholino ligand.

13. A drug delivery system comprising:
a drug coupled to a morpholino ligand comprising Formula I that specifically binds a target, wherein Formula I is: 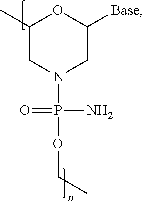
Base is any natural or non-natural base, and n is an integer between 2 and 200, wherein the morpholino ligand specifically binds the target via non Watson-Crick binding.

14. The drug delivery system of claim 13, wherein the target is a protein.

15. The drug delivery system of claim 14, wherein the protein is located on the surface of a cell.

16. The drug delivery system of claim 13, wherein the drug is a chemotherapeutic, an analgesic, or an anti-inflammatory.

16. A labeling system comprising:
a label coupled to a morpholino ligand comprising Formula I that specifically binds a target, wherein Formula I is: 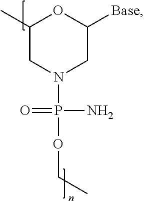
Base is any natural or non-natural base, and n is an integer between 2 and 200, wherein the morpholino ligand specifically binds the target via non Watson-Crick binding.

18. The label system of claim 17, wherein the target is a protein.

19. The label system of claim 18, wherein the protein is located on the surface of a cell.

20. The label system of claim 17, wherein the label is a fluorescent label, a quantum dot, or a radiopaque marker.
